(19) United States
(12) Patent Application Publication
(10) Pub. No.: US 2003/0198987 A1
(43) Pub. Date: Oct. 23, 2003

(54) Methods for designing oligo-probes with high hybridization efficiency and high antisense activity

(76) Inventor: Olga V. Matveeva, Salt Lake City, UT (US)

Correspondence Address: Alison B. Mohr, Parsons Behle & Latimer, Suite 1800, 201 South Main Street, Salt Lake City, UT, 84111-2218, US

(21) Appl. No.: 10/374,253

(22) Filed: Feb. 26, 2003

Related U.S. Application Data
(60) Provisional application No. 60/360,888, filed on Feb. 28, 2002.

(51) Int. Cl. C12Q 1/68; G06G 7/48; G06G 7/58; G06F 19/00; G01N 33/48; G01N 33/50

(52) U.S. Cl. 435/6; 702/20; 703/11

(57) ABSTRACT

Methods are disclosed for selecting oligonucleotide probes with high target affinity. These methods include combining several types of thermodynamic criteria or nucleotide composition criteria for the selection of efficient oligonucleotide probes. The development of these methods was based on statistical analysis of datasets of hybridization or antisense experiments. The methods described here demonstrated variable efficiency in practice, however any or all of them can be used depending on the task of probe design.

### PRIORITY CLAIM

This application claims priority to U.S. Provisional Application Serial No. 60/360,888 filed Feb. 28, 2002 entitled “Method for Designing Oligonucleotides with High Antisense Activity and High Hybridization Efficiency,” which is incorporated herein by reference for all purposes in its entirety.

### FIELD

The inventions relate to the field of oligo-probe design. Specifically, the design of oligo-probes with high target affinity and/or antisense efficiency.

### BACKGROUND

Many techniques of molecular biology require interaction of oligonucleotides with targets as a basic step in their procedure. Examples include current amplification techniques that employ polymerase chain reaction (PCR, Roche Molecular Systems); ligase chain reaction (LCR, Abbott Laboratories); nucleic acid sequence-based amplification (NASBA, Organon Teknika NV); strand displacement amplification (SDA, Becton Dickinson); transcription-mediated amplification (TMA, Gen-Probe, Inc.); oligonucleotide array gene expression monitoring (high density oligonucleotide arrays, Affymetrix, Inc.) and antisense mediated down-regulation of gene expression (ISIS Pharmaceuticals, Inc.). References for the oligonucleotide array technology and antisense technology include Lockhart, D. J., Dong, H., Byrne, M. C., Follettie, M. T., Gallo, M. V., Chee, M. S., Mittman, M., Wang, C, Kobayashi, M, Horton, H. and Brown, E. L. (1996) Nat Biotechnol, 14(13), 1675-80; Wodicka, L., Dong, H., Mittman, M., Ho, M. H. and Lockhart, D. J. (1997) Nat Biotechnol, 15(13), 1359-67; and Crooke, S. T. and Dekker, M. (2001) Antisense Drug Technology: Principles, Stratagies and Applications, edited by Stanley T. Crook and Marcel Dekker.

Potential applications for these techniques are very broad, including pathogen detection in clinical medicine, genetic screening and diagnosis, dental and veterinary medicine, drug resistance and susceptibility testing, pharmacogenetic analysis, food testing, and forensic analysis. Efficient interaction between an oligonucleotide probe and a target is crucial for successful application of nucleic acid probes to such diverse fields.

The thermodynamics of oligonucleotide helix formation have been well studied. Equations have been developed that can predict the melting temperature of matching sequences. These equations are based on a two state model (bound and unbound states) using nearest-neighbor thermodynamics. Nearest-neighbor refers to the use of consecutive base pairs as the primary basis on which enthalpy calculations are made (i.e. every possible pair of consecutive bases has its own set of constants for the equations).

However, this two state model does not consider competing structures that can form during oligo-target interaction. The stabilities of competing structures, including the oligonucleotide intra- and inter-molecular self-interactions as well as the target RNA structure are important factors that can strongly influence the efficiency of the oligo-target interaction. Therefore, melting temperature predictions as well as hybridization temperature predictions can be made dramatically more accurate if the thermodynamics of competing structure are taken into account.

An equation was proposed (Mathews, D. H., et al. (1999) RNA, 5, 1458-1469) for calculation of summation Gibbs Free Energy change relevant to all possible competing structure formations or melting during the process of oligo-RNA target interactions. The question has not yet been employed for predicting the hybridization behavior of the oligo-probes during array gene expression experiments or during antisense mediated gene-down regulation. In other words, the calculation of theoretical values of Gibbs Free Energy changes have not been used for practical tasks and challenges of molecular probe applications. One of the problems of the matching theory and practice in this case is related to the difficulty in weighting coefficients for Gibbs Free Energy change components involved in the equation. These weighting coefficients are related to the concentrations of molecular structures and their complexes that are mainly unknown in practice. Some embodiments of the inventions advance a new way of finding these weighting coefficients. They also provide a way of employing weighting coefficients for calculating thermodynamic thresholds in Gibbs Free Energy changes that are directly relevant to the categorization of oligo-probes into groups of efficient and non-efficient binders. Some embodiments of the inventions make it possible to use the equation suggested before (Mathews, D. H., et al. (1999) RNA, 5, 1458-1469) for reliable prediction of hybridization properties of oligo-probes. Consequently, it is important to select oligonucleotides that can efficiently interact with their complementary sequence.

The assumption that the oligo-probe and the target are single stranded molecules or a double stranded duplex led to the development of the two-state transition model. This model can be helpful for the calculation of the melting temperatures of the oligo-target duplexes. The values of binding efficiency of the oligo-probes towards a target may correlate with the values of melting temperature of the oligo-target duplexes. Therefore, the two state model is useful for the prediction of the binding efficiency of the oligo-probes toward a target. However, this model does not consider competing structures that can form during oligo-target interaction. The stabilities of competing structures, including the oligonucleotide intra- and inter-molecular self-structures as well as the target RNA structure, are important factors that can strongly influence the efficiency of the oligo-target interaction.

RNA secondary structure and the development of methods that can locate single-stranded regions in the RNA molecules attracted significant efforts. Sczakiel, G. and M. Tabler (1997) Methods Mol Biol., 74, 11-15; Patzel, V., et al. (1999) Nucleic Acids Res., 27, 4328-4334; Lehmann, M. J., Patzel, V. and Sczakiel, G. (2000) Nucleic Acids Res., 28, 2597-2604; Scherr, M., et al. (2000) Nucleic Acids Res., 28, 2455-2461; Sczakiel, G. (2000) Front Biosci., 5, D194-201; and Ding, Y. and C. E. Lawrenece (2001) Nucleic Acids Res., 29, 1034-1046. There is some experimental evidence that oligonucleotides designed to target non-structured RNA regions are indeed frequently efficient in down regulation of particular gene products. Sczakiel, G. and M. Tabler (1997) Methods Mol Biol., 74, 11-15; Patzel, V., et al. (1999) Nucleic Acids Res., 27, 4328-4334; Lehmann, M. J., Patzel, V. and Sczakiel, G. (2000) Nucleic Acids Res., 28, 2597-2604; Scherr, M., et al. (2000) Nucleic Acids Res., 28, 2455-2461; and Sczakiel, G. (2000) Front Biosci., 5, D194-201. It is not known how much oligonucleotide self-pairing decreases binding efficiency.

Software for calculating thermodynamic properties of oligonucleotide structure, target structure and duplex formation has been developed. Mathews, D. H., et al. (1999) RNA, 5, 1458-1469. However, statistical correlations between these thermodynamic properties and oligonucleotide binding efficiency for large datasets of antisense experiments and hybridization data have not yet been reported.

### SUMMARY

Methods are provided for combining several types of thermodynamic criteria or nucleotide composition criteria for the selection of efficient oligonucleotide probes (“oligo-probes”). The development of these methods is based on statistical analysis of datasets of hybridization and antisense experiments. Almost all of these methods include three main procedures: learning, testing and filtering. The methods described herein demonstrate variable efficiency in practice. Any or all of them can be used depending on the task of probe design.

One embodiment of the inventions presents a method of determining predictor values employing weighted thermodynamic parameters such as ΔG_{oligo-target}, ΔG_{oligo-intra}, ΔG_{oligo-inter} and ΔG_{target structure}.

Another embodiment of the inventions presents a method of determining predictor values employing weighted thermodynamic parameters such as ΔG_{oligo-target}, ΔG_{oligo-intra}, and ΔG_{oligo-inter} without consideration of ΔG_{target structure}.

Another embodiment of the inventions presents a method of determining predictor values employing weighted thermodynamic parameters such as ΔG_{oligo-target} and ΔG_{oligo-intra} without consideration of ΔG_{target structure} or oligo-oligo bimolecular interaction.

Yet another embodiment of the inventions presents a method for determining efficient oligo-target binders or antisense molecules with known optimal filtration cut-off points in values of ΔG_{oligo-target}, ΔG_{oligo-intra} and ΔG_{oligo-inter} for selection. The oligo-probes should be selected with ΔG⁰_{37 oligo-target}≦−30 kcal/mol, ΔG⁰_{37 oligo-intra}≧−1.1 kcal/mol, and ΔG⁰_{37 oligo-inter}≧−8 kcal/mol.

Another embodiment of the inventions presents a method of determining predictor values employing weighted proportions of nucleotides in the oligo-probes.

### BRIEF DESCRIPTION OF DRAWINGS

FIG. 1 depicts a summary of the hybridization experiments that were performed to obtain the Agilent Technology (AT) dataset and the Oxford Gene Technology (OGT) dataset.

FIG. 2 depicts an embodiment of the inventions showing the learning, testing and filtration procedures for design of oligonucleotides that can efficiently interact with a target.

FIG. 3 depicts an embodiment of the inventions that includes a method of determining predictor ΔG_{combined} values by employing weighted thermodynamic parameters such as ΔG_{oligo-target}, ΔG_{oligo-intra}, ΔG_{oligo-inter} and ΔG_{target structure}.

FIG. 4 depicts weighting and correlation coefficients for the regression models for the AT and OGT datasets. These regression models include thermodynamic parameters such as ΔG_{oligo-target}, ΔG_{oligo-intra}, ΔG_{oligo-inter} and ΔG_{target structure} as the independent variables and natural logarithmic values of hybridization intensity as dependent variables. The top portion of the table shows weighting and correlation coefficients that were obtained during the learning procedure. The bottom part of the table shows weighting coefficients that were used for the testing procedure and correlation coefficients that were obtained during the testing procedure.

FIG. 5 depicts a correlation and categorization analysis for oligo-probe design tested with the AT dataset as performed in Example 1. The learning procedure was performed with the OGT dataset.

FIG. 6 depicts a correlation and categorization analysis for oligo-probe design tested with the AT dataset as performed in Example 2. The learning procedure was performed with the OGT dataset.

FIG. 7 depicts a correlation and categorization analysis for oligo-probe design tested with the AT dataset as performed in Example 3. The learning procedure was performed with the OGT dataset.

FIG. 8 depicts the efficiency of filtration of antisense oligo-probes applying three filters for the values of ΔG_{oligo-target}, ΔG_{oligo-intra} and ΔG_{oligo-inter}. Filtration was tested employing the WEB dataset.

FIG. 9 depicts an embodiment of the inventions that includes a method of determining predictor values (composition score) by employing weighted proportions of A, G, C and T for each oligo-probe.

FIG. 10 depicts weighting and correlation coefficients for the regression model for the AT and OGT datasets. This regression model includes proportions of A, G, C and T in each oligo-probe as the independent variables and natural logarithmic values of hybridization intensity as the dependent variables. The top portion of the table shows weighting and correlation coefficients that were obtained during the learning procedure. The bottom part of the table shows weighting coefficients that were used for the testing procedure and correlation coefficients that were obtained during testing procedure.

FIG. 11 depicts a correlation and categorization analysis for a method of oligo-probe design tested with the AT dataset as performed in Example 5.

FIG. 12 depicts weighting and correlation coefficients for the regression models for the WEB and ISIS datasets. This regression model includes proportions of A, G, C and T in each oligo-probe as the independent variables and natural logarithmic values of antisense efficiency as the dependent variables. The top portion of the table shows weighting and correlation coefficients that were obtained during the learning procedure. The bottom part of the table shows weighting coefficients that were used for the testing procedure and correlation coefficients that were obtained during the testing procedure.

FIG. 13 depicts a correlation and categorization analysis for a method of oligo-probe design tested with WEB dataset as performed in Example 6.

### DETAILED DESCRIPTION

Definitions

Oligonucleotide: is a short molecule of DNA or RNA that can be chemically modified to improve its stability, target binding ability or other features that are related with its successful application. These modifications include chemical change of the backbone for creation of PNA (protein nucleic acids). Each oligonucleotide is a chain of smaller molecules, called nucleotide residues that are attached to each other.

RNA: is an in vivo or in vitro synthesized molecule of ribonucleic acid.

DNA: is an in vivo or in vitro synthesized molecule of deoxyribonucleic acid.

Target: is a molecule of RNA or DNA. Target includes virtual targets.

Duplex: is a complex formed by an oligonucleotide and a target based on complimentary interaction.

Composition score: is a sum of weighted proportions of the nucleotides in an oligo-probe.

Abbreviations

ΔG_{oligo-target} is an abbreviation for Gibbs Free Energy change related to duplex formation of the oligonucleotide and a target sequence.

ΔG_{oligo-inter} is an abbreviation for Gibbs Free Energy change related to the formation of inter-molecular oligonucleotide self-structure. In other words it is an abbreviation for Gibbs Free Energy change related to the formation of oligo-oligo bimolecular complex.

ΔG_{oligo-intra} is an abbreviation for Gibbs Free Energy change related to the formation of intra-molecular oligonucleotide self-structure.

ΔG_{target structure} is an abbreviation for Gibbs Free Energy change related to the stability of local target structure.

ΔG_{combined} is the sum of weighted Gibbs Free Energy changes described above.

ΔG°_{T} is a general abbreviation for Gibbs Free Energy change related to temperature.

A—is an abbreviation for the adenosine.

G—is an abbreviation for the guanosine.

C—is an abbreviation for the cytidine.

T—is an abbreviation for the thymidine.

Experimental Datasets

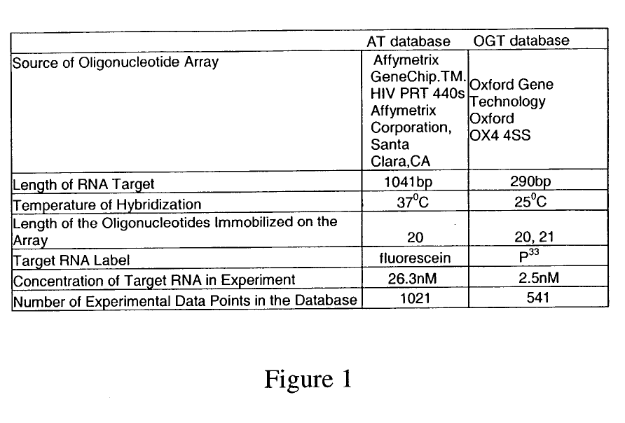

Two datasets of array hybridization experiments were used for oligonucleotide analysis. The comparative characteristics of these datasets are shown in FIG. 1. One of these datasets was taken from U.S. Pat. No. 6,251,588 (Agilent Technology or “AT dataset”). The other was kindly provided by Dr. Verhoef (Oxford Gene Technology or “OGT dataset”). Oligonucleotide scanning arrays provide an approach to monitor the efficiency of hybridization simultaneously for many, or all, target regions of a particular RNA. Target affinity can also be monitored for oligonucleotides of different lengths and self-structures in a single hybridization experiment. Sohail, M., Akhtar, S. and Southern, E. M. (1999) RNA, 5(5), 646-55; Solhail, M. and Southern, E. M. (2001) Methods Mol Biol, 170, 181-99; Sohail, M., Hochegger, H., Klotzbucher, A., Guellec, R. L., Hunt, T. and Southern, E. M. (2001) Nucleic Acids Res, 29(10), 2041-51; Southern, E. M., Case-Green, S. C., Elder, J. K., Johnson, M., Mir, K. U., Wang, L. and Williams, J. C. (1994) Nucleic Acids Res, 22(8), 1368-73; Southern, E. M., Mir, K. and Shchepinov, M. (1999) Nat Genet, 21 (1 Suppl), 5-9; Southern, E. M. (2001) Methods Mol Biol, 170,1-15; and Williams, J. C., Case-Green, S.C., Mir, K. U. and Southern, E. M. (1994) Nucleic Acids Res, 22(8), 1365-7; U.S. Pat. Nos. 5,700,637 (E. Southern) and 5,667,667 (E. Southern). This technology is useful for studying oligonucleotide-related factors that influence the ability of an oligonucleotide to hybridize with RNA.

In addition, two datasets were used for antisense oligonucleotide analysis.

The first dataset (WEB dataset) includes data from antisense oligonucleotide screening experiments reported in literature. Giddings, M. C., et al. (1999) Bioinformatics, 16, 843-844. This dataset is available on the WEB at http://antisense.genetics.utah.edu. The second dataset (ISIS dataset) utilizes data from experiments performed at Isis Pharmaceuticals and has not yet been reported in literature. These datasets include activity values and antisense oligonucleotide sequences. Activity value is expressed as the ratio of a level of a particular mRNA or protein measured in cells after treatment with the experimental antisense oligonucleotides versus control oligonucleotides. There are 316 oligonucleotides in the WEB dataset and 908 in the Isis dataset.

The datasets used in the examples below are merely for illustration and in no way meant to limit the claims. Alternative hybridization and antisense datasets may be used in some embodiments of the inventions. In addition, it is not necessary that the first and second datasets be from different sources. For example, a first and second dataset may be derived from the same original dataset source, by dividing the original source into two discrete datasets that do not overlap.

Software for Thermodynamic and Statistical Analysis

Thermodynamic properties for antisense oligonucleotides and relevant duplexes were calculated using the programs OligoWalk from RNAstructure 3.5 (http://128.151.176.70/RNAstructure.html) and OligoScreen. OligoWalk predicts the equilibrium affinity of complementary oligonucleotides to a target. Mathews, D. H., et al. (1999) RNA, 5, 1458-1469. This program considers the predicted stability of the oligo-target duplex and the competition with predicted secondary structure of both the target and the oligonucleotide. Both inter- and intra-molecular oligonucleotide self-structure are considered at a user-defined concentration.

OligoScreen considers only the predicted stability of the oligo-target duplex and the competition with predicted secondary structure of the oligonucleotide, without consideration of RNA secondary structure. For determination of ΔG°₃₇, both programs use thermodynamic parameters for the nearest neighbor model. Sugimoto, N., et al. (1995) Biochemistry, 34, 11211-11216; Santa Lucia, J., Jr., Allawi, H. T., and Seneviratne, P. A. (1996) Biochemistry, 35, 3555-3562; Allawi, H. T. and Santa Lucia, J., Jr. (1997) Biochemistry, 36, 10581-10594; Santa Lucia, J., Jr. (1998) Proc Natl Acad Sci USA, 95, 1460-1465; and Xia, T., et al. (1998) Biochemistry, 37, 14719-14734.

The programs mentioned above calculate thermodynamic parameters at 37° C. Some array hybridization experiments were performed at 25° C. OligoAnal (Microsoft Excel Macro) was created to achieve consistency between experimental data and theoretical calculations. This macro can produce evaluations of the ΔG_{oligo-target} and ΔG_{oligo-inter} for each analyzed oligonucleotide at any temperature using thermodynamic parameters for the nearest neighbor model.

The program mfold version 3 at http://www.bioinfo.rpi.edu/applications/mfold/old/rna/form4.cgi was used for calculation of ΔG°_{T} values associated with oligonucleotide intra-molecular pairing potentials. Nucleic acid conformation was assumed to be linear, the temperature of hybridization was 25° C. and the ionic conditions were 1 M Na^{+. In the program output the positive values of ΔG°}₅ were changed to 0.

Statistical tools from Excel (Microsoft, Inc) were used for sorting, scatter-plot data presentations and regression analysis. The regression analysis was used to perform weight assignment for independent variables. It employed a “least squares” method to fit a line through a set of observations.

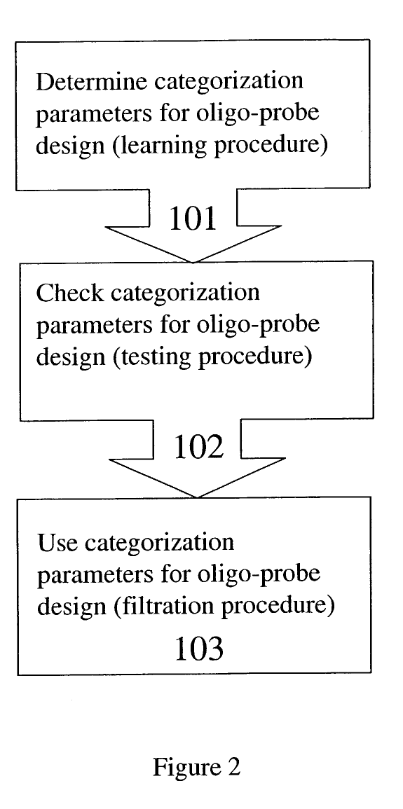

FIG. 2 illustrates the three main procedures used in some embodiments of the inventions including the methods of: learning 201, testing 202 and filtering 203. The goal of the learning procedure 201 is to find a reliable and convenient predictor of the behavior of oligo-probes by analyzing a first dataset. The learning procedure 201 includes the steps of determining parameters that are contributing to the predictor values and steps of determining weights for these parameters. It also includes the step of detection of the threshold in the predictor values. This threshold should be employed for the categorization of oligo-probes into groups of efficient and non-efficient target binders. The goal of the testing procedure 202 is to determine how reliable this predictor is by checking it against a second dataset. The testing procedure 202 includes the step of categorization of the oligo-probes according to their predictor values. If this categorization is successful, then the filtering procedure 203 can be used for oligo-probe design. The goal of the filtering procedure 203 is to categorize the candidate oligo-probes according to their predictor values and to select a group for oligo-probe design. This selected group should include efficient target binders.

### EXAMPLE 1

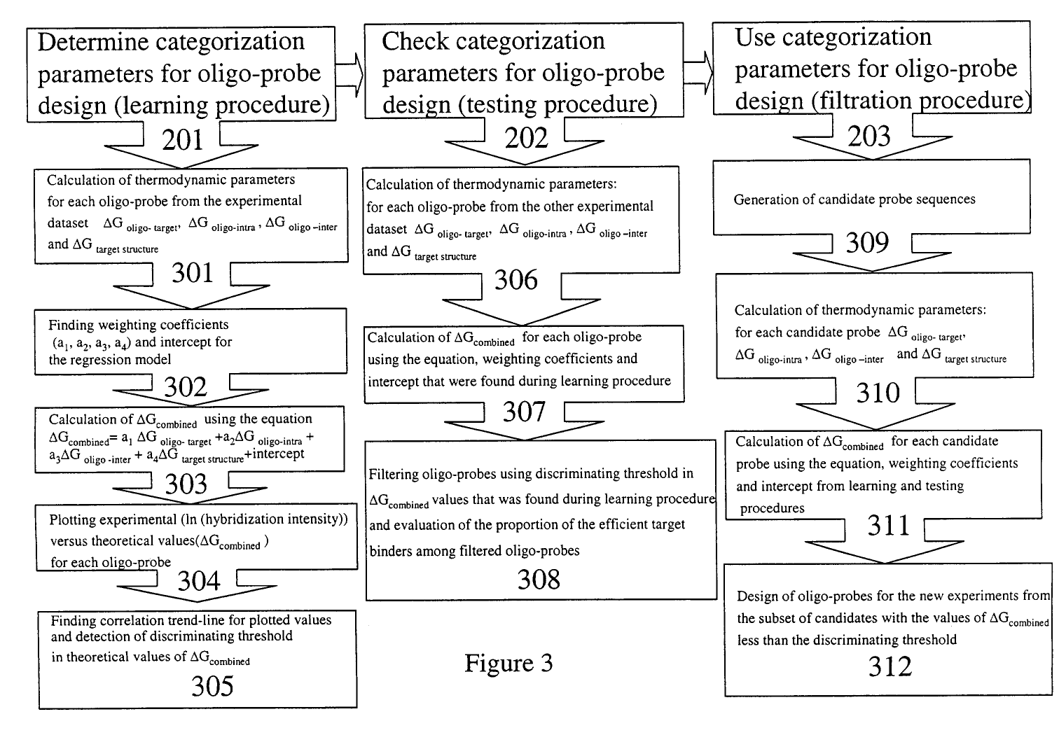

FIG. 3 depicts an embodiment of the inventions that includes a method of determining predictor ΔG_{combined} values employing weighted thermodynamic parameters such as ΔG_{oligo-target}, ΔG_{oligo-intra}, ΔG_{oligo-inter} and ΔG_{target structure}.

Learning Procedure for the Array Hybridization Dataset.

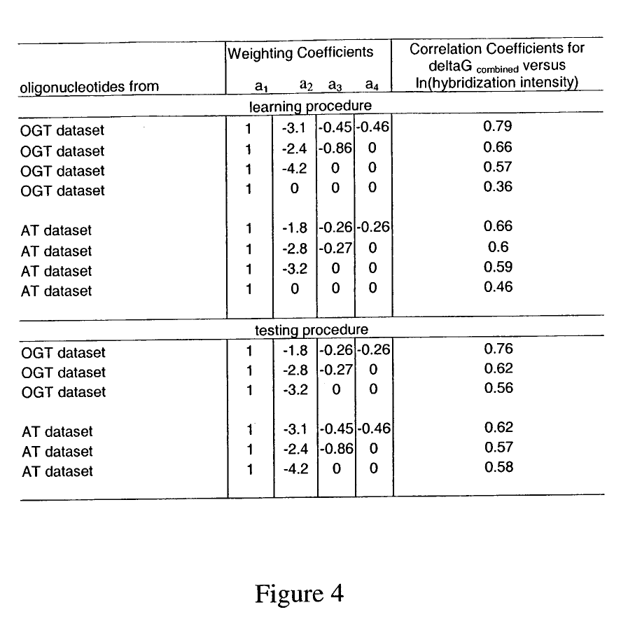

The thermodynamic parameters for each oligonucleotide from the array hybridization (“OGT”) dataset including ΔG_{oligo-target}, ΔG_{oligo-intra}, ΔG_{oligo-inter} and ΔG_{target structure} were calculated 301. The intercept and weighting coefficients (a₁, a₂, a₃, and a₄) for the regression model equation with the values of ΔG_{oligo-target}, ΔG_{oligo-intra}, ΔG_{oligo-inter} and ΔG_{target structure} as the independent variables were found employing a data analysis tool from Excel 302. Natural logarithmic values of intensity of the hybridization for each oligo-probe were calculated for the regression analysis. These values, as listed in FIG. 4, were used for the Y input range, while the values of ≢G_{oligo-target}, ΔG_{oligo-intra}, ΔG_{oligo-inter} and ΔG_{target structure} were used for the X input range. The values of weighting coefficients and intercept were normalized towards a₁. The ΔG_{combined} values were calculated for each oligonucleotide in the dataset using the equation ΔG_{combined}=a₁ΔG_{oligo-target}+a₂ΔG_{oligo-intra}+a₃ΔG_{oligo-inter}+a₄ΔG_{target structure}+intercept 303. The logarithmic values of hybridization efficiency were plotted versus calculated values of ΔG_{combined} values for each oligo-probe 304. The oligo-probes were categorized into groups of efficient RNA binders and non-efficient RNA binders with an arbitrary chosen cut-off point. The discriminating threshold in values of ΔG_{combined} was found. The oligo-probes were categorized according to this discriminating threshold 305. The oligo-probes with normalized values of ΔG_{combined}≦−23 kcal/mol were grouped into a first subset. The oligo-probes with normalized values of ΔG_{combined}>−23 kcal/mol were grouped into a second subset. The proportion of oligo-probes with high hybridization efficiency was determined in the filtered subset (first subset) and in the second subset.

Testing Procedure for Array Hybridization Dataset.

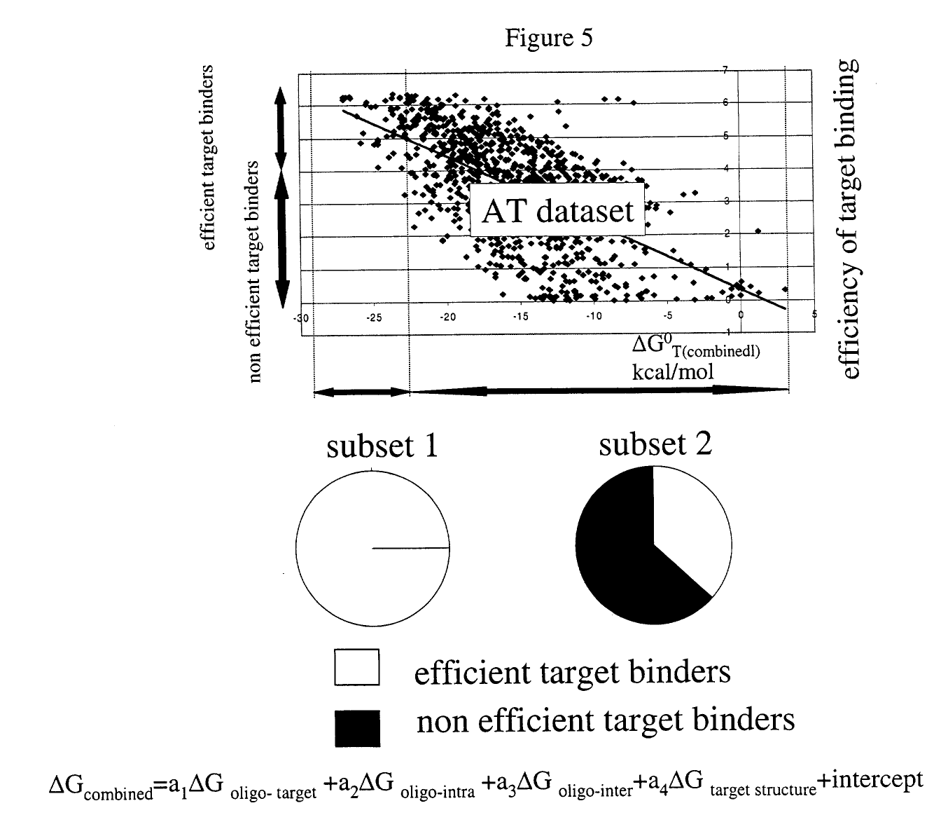

The thermodynamic parameters for each oligonucleotide from the AT dataset such as ΔG_{oligo-target}, ΔG_{oligo-intra}, ΔG_{oligo-inter} and ΔG_{target structure} were calculated 306. The ΔG_{combined} values were calculated for each oligonucleotide in the dataset using the equation ΔG_{combined}=a₁ΔG_{oligo-target}+a₂ΔG_{oligo-intra}+a₃ΔG_{oligo-inter}+a₄ΔG_{target structure}+intercept 307. The intercept and weighting coefficients (a₁, a₂, a₃, and a₄) for this equation were taken from the results of the analysis of the OGT dataset. The natural logarithmic values of hybridization efficiency were plotted versus calculated ΔG_{combined} values for each oligo-probe as illustrated in FIG. 5. The discriminating threshold in the values of ΔG_{combined} that was calculated for the OGT dataset was applied for the filtering procedure 203 of the oligo-probes in the AT dataset 308. The oligo-probes with normalized values of ΔG_{combined}≦−23 kcal/mol were grouped into subset 1 of FIG. 5. The oligo-probes with normalized values of ΔG_{combined}>−23 kcal/mol were grouped into subset 2 of FIG. 5. The proportion of oligo-probes with high hybridization efficiency was determined in the filtered subset (subset 1) and in the remaining subset (subset 2).

Filtering Procedure.

If the testing procedure 202 is successful, the oligo-probe candidates are filtered using the predictor threshold that was determined during the learning procedure 201. The steps of the filtering procedure 203 for this example include generation of candidate probe sequences 309, calculation of thermodynamic parameters for each oligo-probe candidate such as ΔG_{oligo-target}, ΔG_{oligo-intra}, ΔG_{oligo-inter} and ΔG_{target structure} 310 and calculation of ΔG_{combined} values for each oligo-probe candidate 311. Finally the oligo-probe candidates are filtered according to the discriminating threshold values of ΔG_{combined}. The oligo-probes for the experiment are designed from the filtered subset 312. FIG. 5 demonstrates that this testing procedure was successful. The efficient RNA binders in the filtered subset (subset 1) represent 100%. Success rate of the filtration is 100%.

### EXAMPLE 2

Learning Procedure for the Array Hybridization Dataset as in Example 1 but Without Consideration of the Target RNA Secondary Structure.

The thermodynamic parameters for each oligonucleotide from the OGT dataset such as ΔG_{oligo-target}, ΔG_{oligo-intra}, and ΔG_{oligo-inter} were taken from the calculation performed in Example 1. The intercept and weighting coefficients (a₁, a₂, and a₃) for the regression model with the values of ΔG_{oligo-target}, ΔG_{oligo-intra}, and ΔG_{oligo-inter} as the independent variables were found employing a data analysis tool from Excel. Natural logarithmic values of intensity of the hybridization for each oligo-probe were calculated for the regression analysis. These values were used for the Y input range, while the values of ΔG_{oligo-target}, ΔG_{oligo-intra}, and ΔG_{oligo-inter} were used for the X input range. The values of weighting coefficients and intercept were normalized towards a₁. The ΔG_{combined} values were calculated for each oligonucleotide in the dataset using the equation ΔG_{combined}=a₁ΔG_{oligo-target}+a₂ΔG_{oligo-intra}+a₃ΔG_{oligo-inter}+intercept. These normalized values for the OGT dataset are shown in FIG. 4. The logarithmic values of hybridization efficiency were plotted versus calculated values of ΔG_{combined} values for each oligo-probe. The oligo-probes were categorized into groups of efficient RNA binders and non-efficient RNA binders with an arbitrary chosen cut-off point in the same method of Example 1. The discriminating threshold in values of ΔG_{combined} was found. The oligo-probes were categorized according to this discriminating threshold. The oligo-probes with normalized values of ΔG_{combined}≦−23 kcal/mol were grouped into a first subset. The oligo-probes with normalized values of ΔG_{combined}>−23 kcal/mol were grouped into a second subset. The proportion of oligo-probes with high hybridization efficiency was determined in the filtered subset (first subset) and in the second subset.

Testing Procedure for the Array Hybridization Dataset.

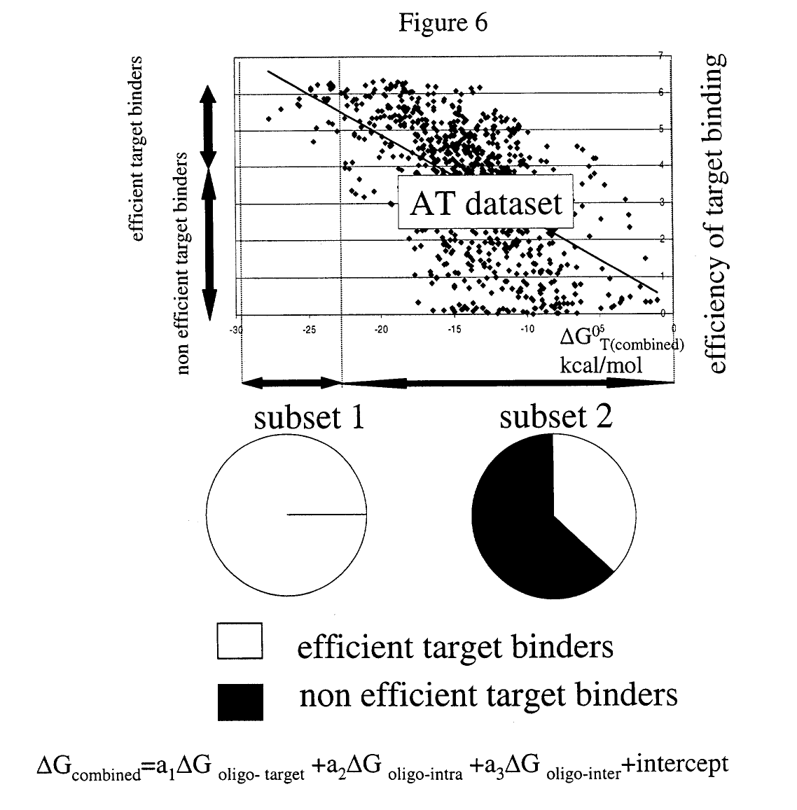

The thermodynamic parameters for each oligonucleotide from the AT dataset such as ΔG_{oligo-target}, ΔG_{oligo-intra} and ΔG_{oligo-inter} were taken from the calculation performed in the Example 1. The ΔG_{combined} values were calculated for each oligonucleotide in the dataset using the equation ΔG_{combined}=a₁ΔG_{oligo-target}+a₂ΔG_{oligo-intra}+a₃ΔG_{oligo-inter}+intercept. The intercept and weighting coefficients (a₁, a₂, and a₃) for this equation were taken from the results of the analysis of the OGT dataset. The natural logarithmic values of hybridization efficiency were plotted versus calculated values of ΔG_{combined} values for each oligo-probe as illustrated in FIG. 6. The discriminating threshold of the values of ΔG_{combined} that was calculated for the OGT dataset was applied for the filtering procedure of the oligo-probes in the AT dataset. The oligo-probes with normalized values of ΔG_{combined}≦−23 were grouped into a subset 1 as illustrated in FIG. 6. The oligo-probes with normalized values of ΔG_{combined}>−23 kcal/mol were grouped into subset 2 as illustrated in FIG. 6. The proportion of oligo-probes with high hybridization efficiency was determined in the filtered subset (subset 1) and in the remaining subset (subset 2).

Filtering Procedure.

If the testing procedure was successful, oligo-probe candidates are filtered using the predictor threshold that was determined during the learning procedure. FIG. 6 demonstrates that this testing procedure was successful. The efficient RNA binders in the filtered subset (subset 1) represented almost 100%. Success rate of the filtration is 100%.

### EXAMPLE 3

Learning Procedure for the Array Hybridization Dataset as in Example 1 but Without Consideration of Oligo-Oligo Bimolecular Interaction or Target RNA Secondary Structure.

The thermodynamic parameters for each oligonucleotide from the OGT dataset such as ΔG_{oligo} target and ΔG_{oligo-intra} were taken from the calculation performed in the Example 1. The intercept and weighting coefficients (a₁ and a₂) for the regression model with the values of ΔG_{oligo-target and ΔG}_{oligo-intra} as the independent variables were found employing a data analysis tool from Excel. Natural logarithmic values of intensity of the hybridization for each oligo-probe were calculated for the regression analysis. These values, as listed in FIG. 4, were used for the Y input range, while the values of ΔG_{oligo-target} and ΔG_{oligo-intra} were used for the X input range. The values of weighting coefficients and intercept were normalized towards a₁. The ΔG_{combined} values were calculated for each oligonucleotide in the dataset using the equation ΔG_{combined}=a₁ΔG_{oligo-target}+a₂ΔG_{oligo-intra}+intercept. The logarithmic values of hybridization efficiency were plotted versus calculated ΔG_{combined} values for each oligo-probe. The oligo-probes were categorized into groups of efficient RNA binders and non-efficient RNA binders with an arbitrary chosen cut-off point by the method of Example 1. The discriminating threshold values of ΔG_{combined} were found. The oligo-probes were categorized according to this discriminating threshold. The oligo-probes with normalized values of ΔG_{combined}≦−27 kcal/mol were grouped into a first subset. The oligo-probes with normalized values of ΔG_{combined}>−27 kcal/mol were grouped into a second subset. The proportion of oligo-probes with high hybridization efficiency was determined in the filtered subset (first subset) and in the second subset.

Testing Procedure for the Array Hybridization Dataset.

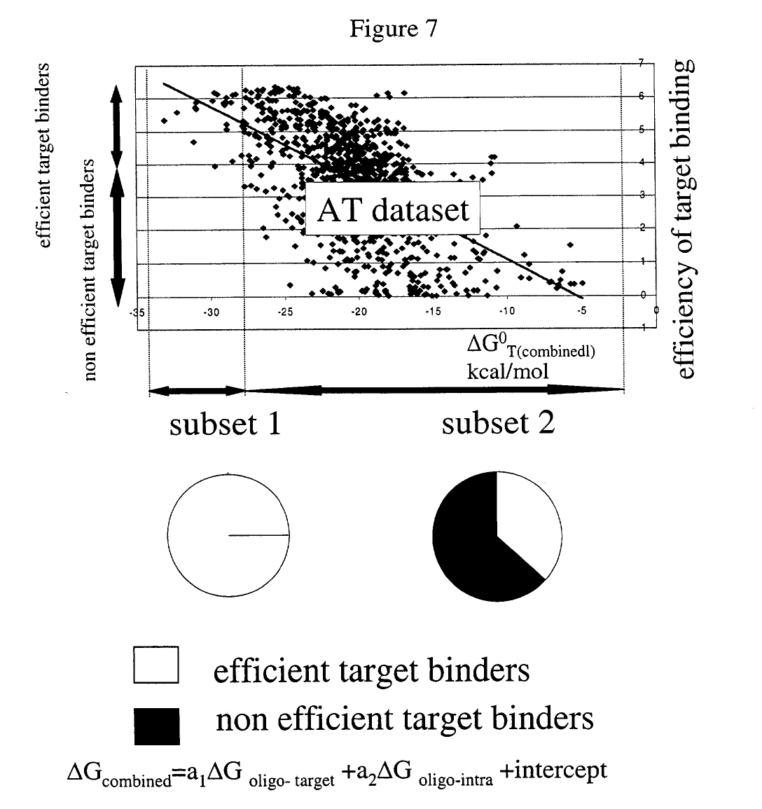

The thermodynamic parameters for each oligonucleotide from the AT dataset such as ΔG_{oligo-target} and ΔG_{oligo-intra} were taken from the calculation performed in Example 1. The ΔG_{combined} values were calculated for each oligonucleotide in the dataset using the equation ΔG_{combined}=a₁ΔG_{oligo-target}+a₂ΔG_{oligo-intra}+intercept. The intercept and weighting coefficients (a₁ and a₂) for this equation were taken from the result of the analysis of the OGT dataset. The natural logarithmic values of hybridization efficiency were plotted versus calculated values of ΔG_{combined} values for each oligo-probe as illustrate in FIG. 7. The discriminating threshold in the values of ΔG_{combined} that was calculated for the OGT dataset was applied for the filtering procedure of the oligo-probes in the AT dataset. The oligo-probes with normalized values of ΔG_{combined}≦−27 kcal/mol were grouped into subset 1 of FIG. 7. The oligo-probes with normalized values of ΔG_{combined}>−27 kcal/mol were grouped into subset 2 of FIG. 7. The proportion of oligo-probes with high hybridization efficiency was determined in the filtered subset (subset 1) and in the remaining subset (subset 2).

Filtering Procedure.

If testing procedure was successful, oligo-probe candidates are filtered using predictor threshold that was determined during learning procedure. FIG. 7 demonstrates that this testing procedure was successful. The efficient RNA binders in the filtered subset (subset 1) represented almost 100%. Success rate of the filtration is 100%.

### EXAMPLE 4

Learning Procedure for Determining Optimal Filtration Cut-Off Points for Prediction of Efficient Antisense Oligonucleotides.

The thermodynamic parameters for each oligonucleotide from the ISIS dataset such as ΔG_{oligo-target}, ΔG_{oligo-intra}, and ΔG_{oligo-inter} were calculated. The oligo-probes were categorized into groups of antisense efficient and non-efficient with an arbitrary chosen cut-off point. The optimal filtering thermodynamic cut off points for ΔG_{oligo-target}, ΔG_{oligo-intra} and ΔG_{oligo-inter} that can be employed for the selection of the antisense efficient oligonucleotides were found using a trial and error approach. The optimal thermodynamic cut off points include −30 kcal mol for ΔG_{oligo-target}, −8 kcal/mol for ΔG_{oligo-inter} and −1.1 kcal/mol for ΔG_{oligo-intra}. It was found that among the oligonucleotides that form stable duplexes with RNA (ΔG°₃₇≦−30 kcal/mol) and have small self-interaction potential, the values for self-interaction should be (ΔG°₃₇)≧−8 kcal/mol, inter-oligonucleotide pairing and (ΔG°₃₇)≧−1.1 kcal/mol intra-oligonucleotide pairing, and the proportion of efficient antisense molecule is much higher than in the total ISIS dataset.

Testing Procedure for Antisense Oligonucleotide Dataset.

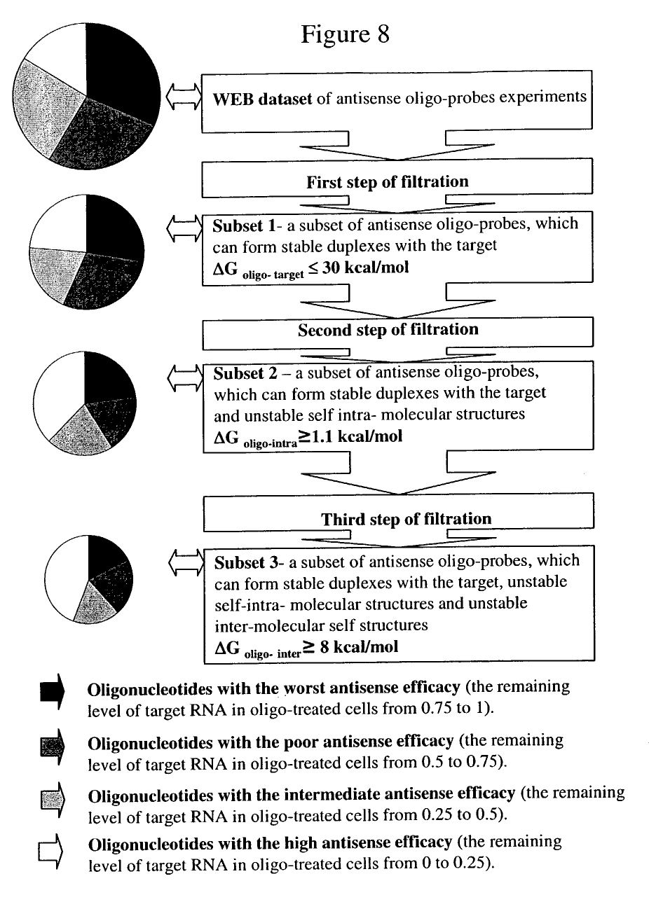

The calculation of ΔG_{oligo-target}, ΔG_{oligo-intra} and ΔG_{oligo-inter} was performed for the oligo-probes from the WEB dataset. The oligo-probes were categorized into groups of antisense efficient and non-efficient with an arbitrary chosen cut-off point that was the same as in the learning procedure. The proportion of antisense efficient molecules was calculated in the subset of the oligo-probes with ΔG⁰_{37 oligo-target}≦−30 kcal/mol, ΔG⁰_{37 oligo-intra}≧−1.1 kcal/mol, and ΔG⁰_{37 oligo-inter}>−8 kcal/mol. FIG. 8 shows that the proportion of the efficient antisense molecules in this filtered subset was much higher than in the initial dataset.

Filtering Procedure.

Success rate of the filtration is 43%.

### EXAMPLE 5

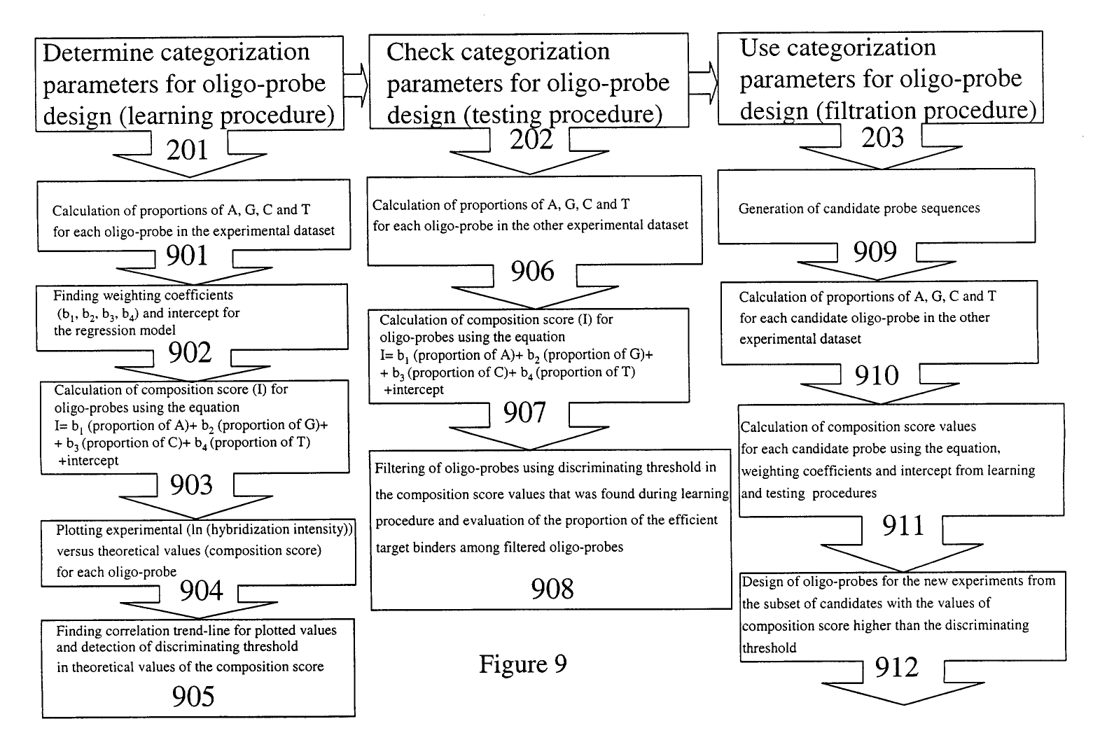

FIG. 9 depicts an embodiment of the inventions that includes a method of determining composition score by employing weighted proportions of A, G, C and T for each oligo-probe.

Learning Procedure for the Array Hybridization Dataset with Consideration of Composition of the Oligo-Probes.

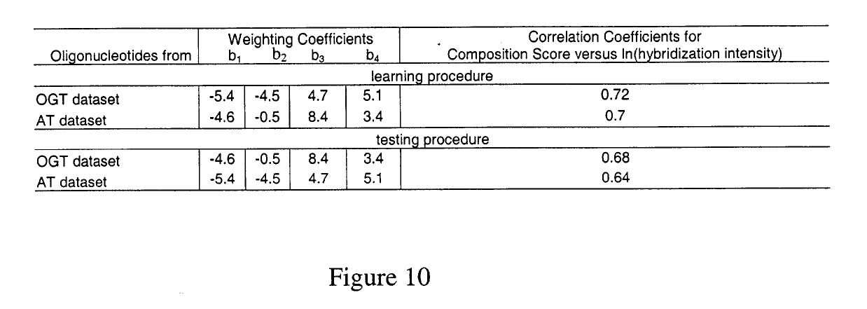

The proportions of A, G, C and T in the sequence of each oligonucleotide from the OGT dataset were calculated 901. The intercept and weighting coefficients (b₁, b₂, b₃, and b₄) for the regression model equation with the values of proportions of A, G, C and T in the sequence of each oligonucleotide as the independent variables were found employing a data analysis tool from Excel 902. Natural logarithmic values of intensity of the hybridization for each oligo-probe were calculated for the regression analysis. These values were used for the Y input range, while the values of proportions of A, G, C and T were used for the X input range. The values of composition score (I) were calculated for each oligonucleotide in the dataset using the equation I=b₁(proportion of A)+b₂(proportion of G)+b₃(proportion of C)+b₄(proportion of T)+intercept 903. These values for the OGT dataset are shown in FIG. 10. The logarithmic values of hybridization efficiency were plotted versus calculated values of composition score for each oligo-probe 904. The oligo-probes were categorized into the groups of efficient RNA binders and non-efficient RNA binders with an arbitrary chosen cut-off point. The discriminating threshold in values of composition score was found 905. The oligo-probes were categorized according to this discriminating threshold. The oligo-probes with a composition score≧6 were grouped into a first subset. The oligo-probes with a composition score<6 were grouped into a second subset. The proportion of oligo-probes with high hybridization efficiency was determined in the filtered subset (first subset) and in the second subset.

Testing Procedure for Array Hybridization Dataset.

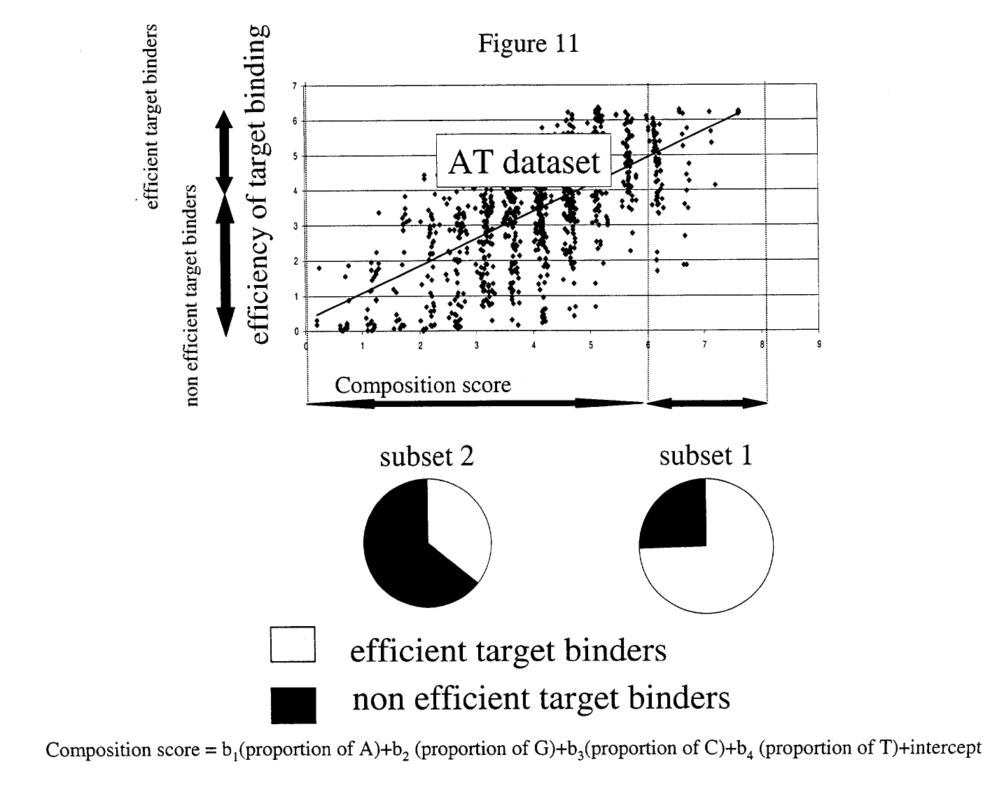

The proportions of A, G, C and T in the sequence of each oligonucleotide from the OGT dataset were calculated 906. The composition score was calculated for each oligo-probe 907. The intercept and weighting coefficients (b₁, b₂, b₃, and b₄) for this equation were taken from the results of the analysis of the OGT dataset. The natural logarithmic values of hybridization efficiency were plotted versus calculated values of composition score for each oligo-probe as shown in FIG. 11. The discriminating threshold in the values of composition score that was calculated for the OGT dataset was applied for the filtering procedure of the oligo-probes in the AT dataset 908. The oligo-probes with a composition score≧6 were grouped into subset 1 of FIG. 11. The oligo-probes with a composition score<6 were grouped into subset 2 of FIG. 11. The proportion of oligo-probes with high hybridization efficiency was determined in the filtered subset (subset 1) and in the remaining subset (subset 2).

Filtering Procedure.

If the testing procedure 202 is successful, oligo-probe candidates are filtered 203 using the predictor threshold that was determined during the learning procedure 201. The steps of the filtering procedure 203 as shown in FIG. 9 include: generation of candidate probe sequences 909, calculation of the proportions of A, G, C and T 910 and calculation of the values of the composition score for each oligo-probe candidate 911. Finally the oligo-probe candidates are filtered according to discriminating threshold values of composition score. The oligo-probes for the experiment can then be designed from the filtered subset 912. FIG. 11 demonstrates that this testing filtration procedure was successful. The efficient RNA binders in the filtered subset (subset 1) represented 75%. Success rate of the filtration is 75%.

### EXAMPLE 6

Learning Procedure for the Antisense Oligonucleotide Dataset with Consideration of Composition of the Oligo-Probes.

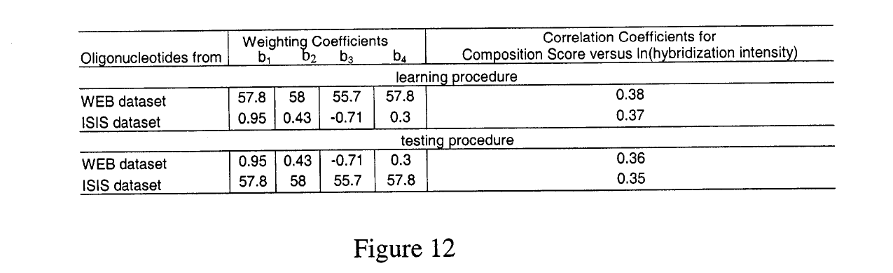

The proportions of A, G, C and T in the sequence of each oligonucleotide from the ISIS dataset were calculated as in Example 5. The intercept and weighting coefficients (b₁, b₂, b₃, and b₄) for the regression model with the values of proportions of A, G, C and T in the sequence of each oligonucleotide as the independent variables were found employing a data analysis tool from Excel. Natural logarithmic values of antisense efficiency for each oligo-probe were calculated for the regression analysis. These values, as listed in FIG. 12, were used for the Y input range, while the values of proportions of A, G, C and T were used for the X input range. The values of composition score (I) were calculated for each oligonucleotide in the dataset using the equation I=b₁(proportion of A)+b₂ (proportion of G)+b₃(proportion of C)+b₄ (proportion of T)+intercept. The logarithmic values of antisense efficiency were plotted versus calculated values of composition score for each oligo-probe. The oligo-probes were categorized into groups of antisense efficient and non-efficient with an arbitrary chosen cut-off point. The discriminating threshold in values of composition score was found. The oligo-probes were categorized according to this discriminating threshold. The oligo-probes with a composition score≦−0.8 were grouped into a first subset. The oligo-probes with a composition score>−0.8 were grouped into a second subset. The proportion of oligo-probes with high antisense efficiency was determined in the filtered subset (first subset) and in the second subset.

Testing Procedure for the Antisense Oligonucleotide Dataset.

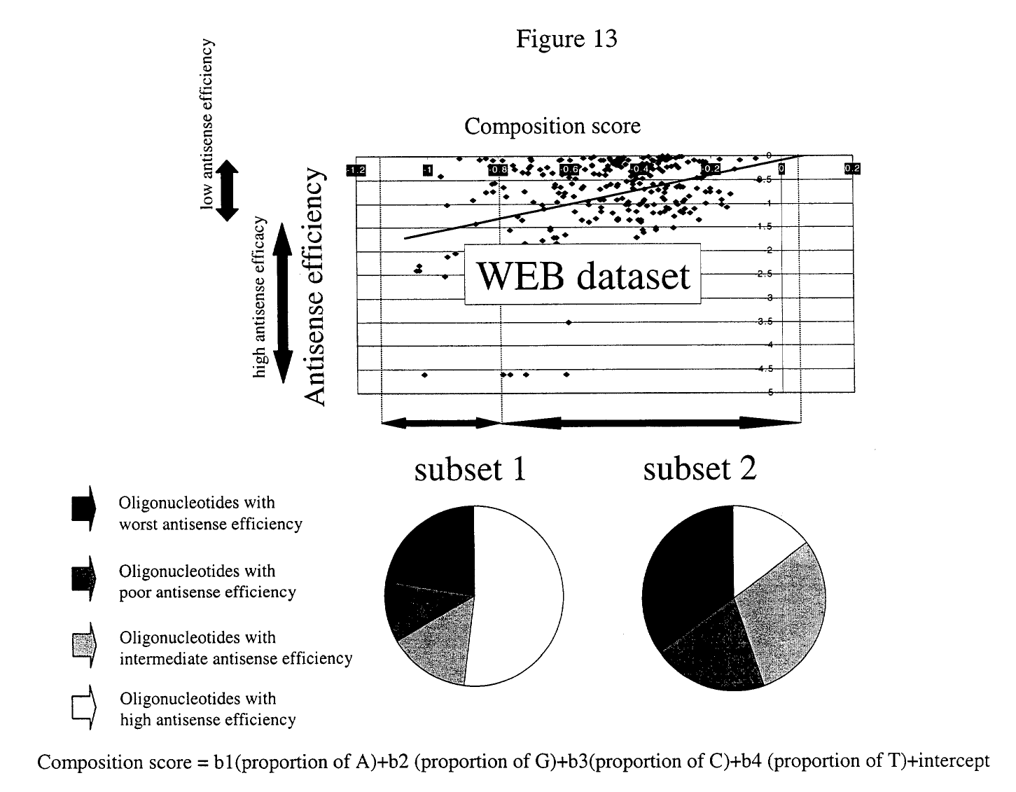

The proportions of A, G, C and T in the sequence of each oligonucleotide from the WEB dataset were calculated. The intercept and weighting coefficients (b₁, b₂, b₃, and b₄) for this equation were taken from the results of the analysis of the ISIS dataset. The natural logarithmic values of antisense efficiency were plotted versus calculated values of composition score for each oligo-probe as illustrated in FIG. 13. The discriminating threshold in the values of composition score that was calculated for the ISIS dataset was applied for the filtering procedure of the oligo-probes in the AT dataset. The oligo-probes with a composition score≦−0.8 were grouped into subset 1 of FIG. 13. The oligo-probes with a composition score>−0.8 were grouped into subset 2 of FIG. 13. The proportion of oligo-probes with high antisense efficiency was determined in the filtered subset (subset 1) and in the remaining subset (subset 2).

Filtering Procedure.

FIG. 13 demonstrates that this testing filtration procedure was successful. In the filtered subset (subset 1) the active antisense molecules represented more than half of oligo-probes. Success rate of the filtration is 57%.

In conclusion, thermodynamic or composition selection criteria can be used for the successful design of oligonucleotides with high target affinity and/or high antisense activity.

The above drawings and examples incorporated in and forming a part of the specification illustrate preferred embodiments of the present inventions. Some, although not all, alternative embodiments are described in the description and therefore the drawings and examples are not intended to limit the scope of the inventions.

1. A method for designing oligo-probes with a high target affinity, said method comprising the steps of:
a. learning;
b. testing; and
c. filtering.

2. The method of claim 1 wherein said learning step further comprises the steps of:
a. calculating thermodynamic parameters for each of the oligo-probes in a first dataset;
b. determining weighting coefficients and an intercept for a regression model equation, wherein natural logarithmic values of hybridization intensity of the oligo-probes toward a target are dependent variables and said thermodynamic parameters are independent variables;
c. calculating Gibbs Free Energy change combined values of said thermodynamic parameters of said first dataset using said weighting coefficients and intercept for said regression model equation;
d. plotting said hybridization intensity versus Gibbs Free Energy change combined values of said first dataset; and
e. determining a correlation trend lines and a discriminating threshold in the values of Gibbs Free Energy change combined.

3. The method of claim 2 wherein said thermodynamic parameters include one or more of the following:
a. a Gibbs Free Energy change related to duplex formation of an oligonucleotide and a target sequence,
b. a Gibbs Free Energy change related to the formation of inter-molecular oligonucleotide self-structure,
c. a Gibbs Free Energy change related to the formation of intra-molecular oligonucleotide self-structure, or
d. a Gibbs Free Energy change related to the stability of a local target structure.

4. The method of claim 3 wherein said testing step further comprises the steps of:
a. calculating said thermodynamic parameters for each of the oligo-probes in a second dataset;
b. calculating Gibbs Free Energy change combined values of said thermodynamic parameters of said second dataset using said regression model equation;
c. categorizing the oligo-probes from said second dataset according to said Gibbs Free Energy change combined values of said second dataset using said discriminating threshold;
d. determining success of said categorizing step.

5. The method of claim 4 wherein said filtering step further comprises the steps of:
a. generating candidate probe sequences;
b. calculating said thermodynamic parameters for each of said candidate probe sequences;
c. calculating Gibbs Free Energy change combined values for each of said candidate probe sequences using said regression model equation;
d. designing a set of oligo-probes including the oligo-probes with said Gibbs Free Energy change combined values of said candidate probe sequences being less than said discriminating threshold.

6. The method of claim 1 wherein said learning step further comprises the steps of:
a. calculating the proportions of A, G, C and T for each of the oligo-probes in a first dataset;
b. determining weighting coefficients and an intercept for a regression model equation, wherein natural logarithmic values of hybridization intensity of the oligo-probes toward a target are dependent variables and said proportions of A, G, C and T are independent variables;
c. calculating composition score values of said first dataset using said weighting coefficients and intercept for said regression model equation;
d. plotting said hybridization intensity versus said composition score values of said first dataset; and
e. determining a correlation trend line and a discriminating threshold in the values of composition score.

7. The method of claim 6 wherein said testing step further comprises the steps of:
a. calculating the proportions of A, G, C and T for each of the oligo-probes in a second dataset;
b. calculating composition score values of said second dataset using said regression model equation;
c. categorizing the oligo-probes from said second dataset using said discriminating threshold;
d. determining success of said categorizing step.

8. The method of claim 7 wherein said filtering step further comprises the steps of:
a. generating candidate probe sequences;
b. calculating the proportions of A, G, C and T for each of said candidate probe sequences;
c. calculating composition score values for each of said candidate probe sequences using said regression model equation;
d. designing a set of oligo-probes including the oligo-probes with said composition score values of said candidate probe sequences being less than said discriminating threshold.

9. A method for designing oligo-probes capable of interacting with a high antisense efficiency, said method comprising the steps of:
a. learning;
b. testing; and
c. filtering.

10. The method of claim 9 wherein said learning step further comprises the steps of:
a. calculating thermodynamic parameters for each of the oligo-probes in a first dataset;
b. determining weighting coefficients and an intercept for said regression model equation, wherein natural logarithmic values of antisense efficiency of the oligo-probes toward a target are dependent variables and said thermodynamic parameters are independent variables;
c. calculating Gibbs Free Energy change combined values of said thermodynamic parameters of said first dataset using said weighting coefficients and intercept for said regression model equation;
d. plotting antisense efficiency versus Gibbs Free Energy change combined values of said first dataset; and
e. determining a correlation trend line and a discriminating threshold in the values of Gibbs Free Energy change combined.

11. The method of claim 10 wherein said thermodynamic parameters include one or more of the following:
a. a Gibbs Free Energy change related to duplex formation of an oligonucleotide and a target sequence,
b. a Gibbs Free Energy change related to the formation of inter-molecular oligonucleotide self-structure,
c. a Gibbs Free Energy change related to the formation of intra-molecular oligonucleotide self-structure, or
d. a Gibbs Free Energy change related to the stability of a local target structure.

12. The method of claim 11 wherein said testing step further comprises the steps of:
a. calculating said thermodynamic parameters for each of the oligo-probes in a second dataset;
b. calculating Gibbs Free Energy change combined values of said thermodynamic parameters of said second dataset using said regression model equation;
c. categorizing the oligo-probes from said second dataset using said discriminating threshold;
d. determining success of said categorizing step.

13. The method of claim 12 wherein said filtering step further comprises the steps of:
a. generating candidate probe sequences;
b. calculating said thermodynamic parameters for each of said candidate probe sequences;
c. calculating Gibbs Free Energy change combined values for each of said candidate probe sequences using said regression model equation;
d. designing a set of oligo-probes including the oligo-probes with said Gibbs Free Energy change combined values less than said discriminating threshold.

14. The method of claim 9 wherein said learning step further comprises the steps of:
a. calculating the proportions of A, G, C and T for each of the oligo-probes in a first dataset;
b. determining weighting coefficients and an intercept for a regression model equation, where natural logarithmic values of antisense efficiency of the oligo-probes toward a target are dependent variables and said proportions of A, G, C and T are independent variables;
c. calculating composition score values of said first dataset using said weighting coefficients and intercept for said regression model equation;
d. plotting antisense efficiency versus said composition score values of said first dataset; and
e. determining a correlation trend line and a discriminating threshold in the values of composition score.

15. The method of claim 14 wherein said testing step further comprises the steps of:
a. calculating the proportions of A, G, C and T for each of the oligo-probes in a second dataset;
b. calculating composition score values of said second dataset using said regression model equation;
c. categorizing the oligo-probes from said second dataset using said discriminating threshold;
d. determining success of said categorizing step.

16. The method of claim 15 wherein said filtering step further comprises the steps of:
a. generating candidate probe sequences;
b. calculating the proportions of A, G, C and T for each of said candidate probe sequences;
c. calculating composition score values for each of the candidate probe sequences using said regression model equation;
d. designing a set of oligo-probes including the oligo-probes with said composition score values of said candidate probe sequences being less than said discriminating threshold.

17. A method for designing oligo-probes with high target affinity for hybridization experiments or antisense efficiency comprising the steps of:
a. calculating thermodynamic parameters for each of a plurality of oligo-probe candidates;
b. filtering said oligo-probe candidates to provide a subset of oligo-probes; and
c. designing a group of oligo-probes that includes said subset of oligo-probes.

18. The method of claim 17 wherein said thermodynamic parameters include one or more of the following:
a. a Gibbs Free Energy change related to duplex formation of an oligonucleotide and a target sequence,
b. a Gibbs Free Energy change related to the formation of inter-molecular oligonucleotide self-structure, or
c. a Gibbs Free Energy change related to the formation of intra-molecular oligonucleotide self-structure.

19. The method of claim 18 wherein said oligo-probes are filtered to provide said subset of oligo-probes by:
a. said Gibbs Free Energy change related to duplex formation of an oligonucleotide and a target sequence being lower than −30 kcal/mol,
b. said Gibbs Free Energy change related to the formation of inter-molecular oligonucleotide self-structure being higher than −8 kcal/mol, and
c. said Gibbs Free Energy change related to the formation of intra-molecular oligonucleotide self-structure being higher than −1.1 kcal/mol.
